(19) United States
(12) Patent Application Publication
(10) Pub. No.: US 2026/0169111 A1
(43) Pub. Date: Jun. 18, 2026

(54) MULTICOMPARTMENT IMAGING USING COMPREHENSIVE MAGNETIC RESONANCE IMAGING PROTOCOL

(71) Applicant: The Regents of the University of California, Oakland, CA (US)

(72) Inventor: Yajun Ma, La Jolla, CA (US)

(21) Appl. No.: 19/420,408

(22) Filed: Dec. 15, 2025

Related U.S. Application Data
(60) Provisional application No. 63/733,638, filed on Dec. 13, 2024.

(51) Int. Cl. G01R 33/56; A61B 5/00; A61B 5/055; G01R 33/48

(52) U.S. Cl. CPC G01R 33/5602; A61B 5/0042; A61B 5/055; G01R 33/4826; G01R 33/4828; G01R 33/5608; A61B 2576/026

(57) ABSTRACT

Disclosed are devices, systems and methods for performing a comprehensive MR imaging protocol to quantify all the major components of the brain, including macromolecules (e.g., myelin lipid, protein, and axonal membranes), myelin water (trapped in the myelin lipid layers) (MW), intracellular/extracellular water (IEW) and free water (FW) (e.g., unrestricted cerebrospinal fluid (CSF) in the subarachnoid space and ventricular system). In some aspects, the MRI method involves the use of magnetization transfer prepared Cones (MT-Cones) sequence for two-pool MT modeling to quantify macromolecular content; short-TR adiabatic inversion-recovery prepared Cones (STAIR-Cones) sequence for myelin water imaging; proton-density weighted Cones (PDw-Cones) sequence for total water imaging; and highly T₂-weighted Cones (T₂w-Cones) sequence for free water imaging.

### STATEMENT REGARDING FEDERALLY SPONSORED RESEARCH OR DEVELOPMENT

This invention was made with government support under AR079484 awarded by the National Institutes of Health. The government has certain rights in the invention.

### CROSS-REFERENCE TO RELATED APPLICATIONS

This patent document claims priority to and benefits of U.S. Provisional Patent Application No. “MULTICOMPARTMENT IMAGING USING COMPREHENSIVE MAGNETIC RESONANCE IMAGING PROTOCOL” and filed Dec. 13, 2024. The entire contents of the aforementioned patent application are incorporated by reference as part of the disclosure of this patent document.

### TECHNICAL FIELD

This patent document relates to magnetic resonance imaging (MRI) technology, and more particularly, to methods of measuring fluid compartments in the brain.

### BACKGROUND

The brain is a complex organ that contains four main tissue and fluid components (also known as fluid compartments), namely motion-restricted macromolecules (MM) (e.g., myelin lipid, myelin basic protein, and axonal membranes with ultrashort T₂s of ˜10 μs), myelin water (MW) (i.e., water trapped in the myelin lipid layers with T₂s of ˜10 ms), intracellular/extracellular water (IEW) (i.e., water semi-restricted within cell cytoplasm and the intercellular space with T₂S ˜40-90 ms), and free water (FW) (e.g., unrestricted cerebrospinal fluid (CSF) in the subarachnoid space and ventricular system with T₂s longer than 1000 ms). Compositional changes in these components occur in many neuroinflammatory and neurodegenerative diseases, such as multiple sclerosis (MS). Magnetic resonance imaging (MRI) has emerged as a critical tool in clinical and research settings due in large part to significant disease-dependent changes in the T₁s and T₂s of tissue and fluid components in the brain.

### SUMMARY

Disclosed are devices, systems and methods for using a comprehensive three-dimensional (3D) magnetic resonance imaging (MRI) protocol to assess major tissue and fluid components in the brain.

The disclosed technology introduces a comprehensive 3D MRI protocol to quantify major tissue and fluid components in the brain, such as macromolecules, myelin water, intra- and extracellular water, and free water. Using four advanced sequences in this protocol, detailed 3D maps are created that show these major tissue and fluid components through imaging and/or quantitative data. Example implementations of the disclosed 3D MRI protocol in both healthy volunteers and patients with multiple sclerosis (MS) were performed, all with a standard MRI machine. The exemplary 3D maps reveal the amounts of macromolecules, myelin water, water inside and outside cells, and free water across different brain regions. Exemplary implementations of the disclosed 3D MRI method were consistent in repeated tests, proving its reliability. In MS patients, macromolecule and myelin water levels were lower in lesions and surrounding areas than in healthy individuals, while free water levels were higher in lesions. The disclosed technology is envisioned to provide critical clinical insights for studying neuroinflammatory and neurodegenerative brain diseases by offering a thorough view of brain tissue health and damage.

In some embodiments in accordance with the disclosed technology, the 3D MRI protocol comprises four different sequences: 1) magnetization transfer prepared Cones (MT-Cones) for two-pool MT modeling to quantify macromolecular content; 2) a short-repetition-rate (short-TR) adiabatic inversion-recovery prepared Cones (STAIR-Cones) for myelin water imaging; 3) proton-density weighted Cones (PDw-Cones) for total water imaging; and 4) highly T₂-weighted Cones (T₂w-Cones) for free water imaging. By integrating these techniques, key brain components-namely macromolecules, myelin water, intra/extracellular water, and free water—can be successfully mapped, which was shown by example implementations of the disclosed method in ten healthy volunteers and five patients with multiple sclerosis (MS) using a 3T clinical scanner. Brain macromolecular proton fraction (MMPF), myelin water proton fraction (MWPF), intra/extracellular water proton fraction (IEWPF), and free water proton fraction (FWPF) values were generated in white matter (WM), gray matter (GM), and MS lesions. Excellent repeatability of the protocol was demonstrated with high intra-class correlation coefficient (ICC) values. In MS patients, the MMPF and MWPF values of the lesions and normal-appearing WM (NAWM) were significantly lower than those in normal WM (NWM) in healthy volunteers. Moreover, significantly higher FWPF values in MS lesions were observed compared to those in NWM and NAWM regions. The example implementations of the disclosed 3D MRI method demonstrate the capability of the disclosed technology to volumetrically map major brain components. The technique may have particular value in providing a comprehensive assessment of neuroinflammatory and neurodegenerative diseases of the brain.

The subject matter described in this patent document can be implemented in specific ways that provide one or more of the following features.

### BRIEF DESCRIPTION OF THE DRAWINGS

FIG. 1 shows plots and diagrams depicting example features of the four sequences in example embodiments of a comprehensive 3D MRI protocol, including MT-Cones, STAIR-Cones, T₂w-Cones, and PDw-Cones, in accordance with the present technology.

FIG. 2 shows a diagram depicting an example workflow to quantify proton fractions of macromolecules, myelin water, intracellular/extracellular water, and free water (e.g., MMPF, MWPF, IEWPF, and FWPF) in the brain, in accordance with the present technology.

TABLE 1 provides detailed sequence parameters used in example implementations of the disclosed 3D MRI protocol, in accordance with the present technology.

FIG. 3 shows MR images representative MT-, PDw-, STAIR-, and highly T₂w-Cones images acquired from a healthy volunteer.

FIG. 4 shows brain multicompartment mapping results, including example MMPF, MWPF, IEWPF, and FWPF maps obtained from the same healthy volunteer featured in FIG. 3.

FIG. 5 shows MR images depicting example proton fraction maps acquired from a normal volunteer and three patients diagnosed with MS.

FIG. 6 shows bar plots illustrating example MMPF, MWPF, IEWPF, and FWPF measurements for the ten normal volunteers and five patients with MS.

TABLE 2 provides a summary of example MMPF, MWPF, IEWPF, and FWPF measurements in normal volunteers and MS patients.

FIG. 7 shows a flow diagram illustrating an example method for characterizing fluid compartments in tissue, in accordance with the present technology.

FIG. 8 shows a diagram of an example embodiment of a system for implementing example embodiments of the 3D MRI protocol, in accordance with the present technology.

### DETAILED DESCRIPTION

Magnetic resonance imaging (MRI) has emerged as a critical tool in clinical and research settings due in large part to significant disease-dependent changes in the T₁s and T₂s of tissue and fluid components in the brain. However, clinical T₁- or T₂-weighted sequences have difficulty distinguishing between pathological processes in lesions such as edema, gliosis, inflammation, demyelination, and remyelination, as well as studying the effects of these pathological processes within each compartment. For example, brain lesions typically appear high signal when imaged with clinical T₂-weighted sequences. This lack of specificity of current clinical sequences limits the value of MRI in disease diagnosis and progression tracking, as well as in treatment monitoring.

Quantitative MRI combined with biophysical models, termed in-vivo histology using MRI (hMRI), has become an attractive technology for non-invasively obtaining structural and compositional information. However, biological tissue systems are very complex, consisting of various types of macromolecules and the environments these macromolecules create for water molecules. This results in different cohorts of water components, each with distinct MRI properties. Therefore, a multicompartment model should be considered to more accurately describe the tissue system. This complexity is further heightened by multiple exchange processes between these macromolecular and water pools, including MT, chemical exchange saturation transfer, and diffusion.

Conventional MRI cannot directly detect signals from macromolecules due to their ultrashort T₂s (˜10 μs). To address this, MT modeling techniques have been developed to indirectly assess structural and compositional changes of macromolecular components in tissues. The two-pool MT model is the most simplified and widely used due to its practicality and reproducibility. In this study, we employed a classic two-pool MT model with RP approximation, to estimate the MMPF in the brain. More recently, researchers have been working hard to address potential biases in traditional MT modeling techniques by incorporating B₁ inhomogeneity correction or expanding the model to include unconstrained T₁ relaxation times and considering contributions from on-resonance saturation and dipolar order. These efforts have successfully improved the accuracy of biological tissue characterization. We expect our proposed multicompartment imaging method to deliver a more reliable estimation of modeling parameters if we incorporate all these improvements into our two-pool MT modeling process.

Exchange processes also play a significant role in non-MT sequences, such as inversion recovery (IR) sequences. A recent ex vivo bovine white matter modeling study found that a bi-component model was required to fit the data acquired by the IR preparation and soft pulse excitation, while a single-component model was sufficient to fit the data with hard pulse excitation. A four-pool model considering intercompartment exchange successfully characterized the white matter tissue system and explained the signal behavior for the different types of IR sequences. This study supports the idea that longitudinal magnetization evolution depends on both intercompartment exchange and spin-lattice relaxation. To explore this further, the apparent T₁ is an aggregate measurement of T₁ relaxation affecting different water components and pertains to all molecules that exchange fast enough with water. Even in intermediate exchange conditions, the long component of the signals measured from water, once the short component (corresponding mostly to exchange) has decayed, contains a mixed contribution from underlying T₁s from all components. The sequence timing, RF saturation, and exchange processes need to be considered when determining the apparent T₁ and disentangling individual T₁s. Otherwise, different sequences may yield significantly different T₁ estimates.

Attempts to improve current clinical sequences have led to many techniques being developed for the assessment of specific brain compartments, and these may improve current approaches used in clinical diagnosis and treatment monitoring. To assess myelin content changes in the brain, several state-of-the-art MW imaging techniques such as multi-compartment T₂/T₂* relaxation measurements and visualization of short transverse relaxation time components (ViSTa) have been developed over the last two decades. The multi-compartment T₂/T₂* relaxation approach models brain tissue with two or more water compartments and separates them by postprocessing based on their T₂ or T₂* differences. In comparison, ViSTa selectively images the short T₁ component (e.g., MW) using a double inversion recovery (DIR) technique without complicated modeling and intensive postprocessing. Brain IEW contents are typically imaged using T₂-weighted and fluid-attenuated inversion recovery (T₂-FLAIR) sequences in the clinical diagnosis of MS. Highly T_{1ρ}- and T₂-weighted sequences have been developed for selective FW imaging.

However, despite the dedicated effort and rapid pace of technological development in this field, there is yet no complete set of 3D protocols available to image and quantify the major tissue and fluid components of the brain in-vivo. Such a protocol could be of considerable value in providing a comprehensive assessment of compositional changes in neuroinflammatory and neurodegenerative disease as well as monitoring therapeutic efficacy. In attempting to achieve this, many techniques and partial solutions have been developed, as described above, but none have offered such a comprehensive set of compatible and effective protocols.

To achieve this goal, we have developed an advanced MRI method using four different sequences: a MT-Cones sequence combined with two-pool MT modeling for MM content estimation, a STAIR-Cones sequence for MW imaging, a proton density-weighted Cones (PDw-Cones) sequence for total water (TW) imaging, and a highly T₂-weighted Cones (T₂w-Cones) sequence for FW imaging. Each sequence can use a 3D Cones acquisition method with a short TE (e.g., 0.8-2 ms), with signal weighting driven by each respective preparation module. By combining the four sequences, we can estimate the proton fractions (PFs) of all the major compartments in the brain (i.e., MMs, MW, IEW, and FW).

In some embodiments of the disclosed methods, the four sequences are combined into a comprehensive protocol: MT-Cones, STAIR-Cones, T₂w-Cones, and PDw-Cones. A Fermi-shaped MT pulse is employed in the MT-Cones sequence to generate MT contrast followed by multispoke Cones data acquisition to speed up the scan. A series of MT-Cones data are acquired with different MT powers and frequency offsets. In addition, brain T₁ values are measured by the recently developed actual flip angle and variable flip angle Cones (AFI-VFA-Cones) technique (sequence diagrams not shown) as an input for two-pool MT modeling. The modified RP MT modeling process is then used to quantify the macromolecular proton fraction (MMPF). The total water proton fraction (TWPF) is calculated as 1-MMPF. With the STAIR-Cones sequence, an Adiabatic Full Passage (AFP) pulse is utilized to invert the longitudinal magnetization of water components without significant compromise due to system B₀ and B₁ inhomogeneities, and this is followed by multispoke Cones data acquisition. Using a sufficiently short repetition time (TR) (e.g., 250 ms) in conjunction with an optimal inversion time (TI) in the STAIR-Cones sequence enables the suppression of signals from long T₂ water components such as IEW and FW with a wide range of T₁s, allowing selective imaging of the fast-recovering short T₁ MW (essentially acting as a T₁ filter).

Additionally, MW has a relatively short T₂ relaxation time (˜10 ms). The longitudinal magnetization of MW is partially inverted by the relatively long AFP pulse (duration=8.64 ms), allowing more signal recovery from MW during TI in STAIR-Cones. The highly T₂ weighted-Cones sequence (T₂w-Cones) includes four major features: (i) a magnetization reset module to generate a constant magnetization recovery, (ii) a T₂ preparation module with a long free decay time (e.g., 350 ms), (iii) a radio frequency (RF) cycling scheme (e.g., the RF phase of the second 90° pulse in the T₂ preparation alternates by 180° in adjacent TRs), and (iv) a variable flip angle (VFA) technique to reduce signal variation along the multispoke data acquisition and improve the signal-to-noise ratio (SNR) performance of the sequence for acquisition of FW signal. The PDw-Cones sequence is utilized for fast TW imaging. It employs a relatively low flip angle (e.g., 1°) for signal excitation and reduction of T₁-weighting. A 3D AFI-Cones sequence was utilized to map and correct B₁ inhomogeneity in the MT modeling. In each acquisition spoke of the above sequences, a slab-selective RF pulse is utilized for signal excitation followed by center-out spiral encoding. This spiral encoding forms a 3D Cones trajectory which covers the whole of k-space efficiently.

The MW, TW, and FW images are directly generated by the STAIR-Cones, PDw-Cones, and highly T₂w-Cones scans, respectively. The MWF, defined as the content ratio between MW and TW, taking into account the mixed T₁ and T₂* weighting terms from the STAIR-Cones and PDw-Cones sequences. The FWF, defined as the content ratio between FW and TW, is calculated in two steps. First, the signal ratio between the highly T₂w-Cones and PDw-Cones is determined. Then, since the FWF in the ventricular region should be 1, the FWF is estimated by normalizing the signal ratio map to the signal ratio from the ventricular region. This normalization step successfully eliminates the mixed T₁, T_{2*}, or T₂ weighting terms from the highly T₂w-Cones and PDw-Cones sequences. With known MWF and FWF, the IEWF, defined as the content ratio between IEW and TW, can easily be obtained. The MMPF is directly obtained by two-pool MT modeling of a series of MT-Cones data. The TWPF can be easily calculated as TWPF=1-MMPF, which is defined as the ratio of TW to total proton content (i.e., TW+macromolecular proton (MMP)).

Finally, with known values of MWF, FWF, and IEWF as well as TWPF, the MWPF, FWPF and IEWPF are obtained.

### Example Embodiments and Example Implementations of the Disclosed Multicompartment Imaging Techniques

Example embodiments and example implementations demonstrating the disclosed multicompartment imaging techniques are described in detail below.

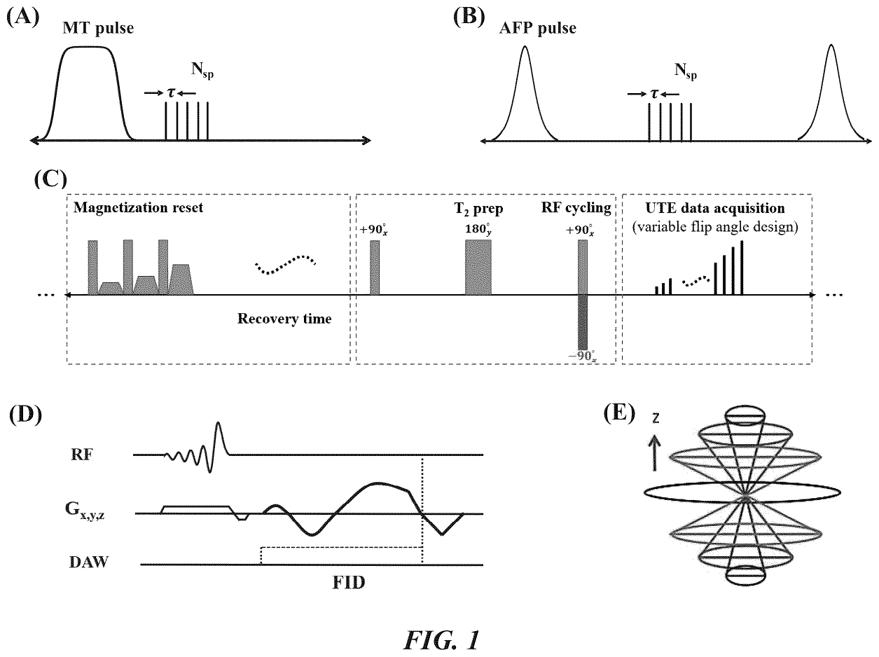

FIG. 1 shows plots and diagrams depicting example features of the four sequences in example embodiments of a comprehensive 3D MRI protocol, including MT-Cones, STAIR-Cones, T₂w-Cones, and PDw-Cones, in accordance with the present technology.

In the MT-Cones sequence, as illustrated in Panel A of FIG. 1, a Fermi-shaped MT pulse can be employed to generate MT contrast. A series of MT-Cones data can be acquired with different MT powers and frequency offsets. In addition, brain T₁ values can be measured by actual flip angle and variable flip angle Cones (AFI-VFA-Cones) technique (sequence diagrams not shown in FIG. 1) as an input for two-pool MT modeling. A 3D AFI-Cones sequence can be utilized to map and correct B₁ inhomogeneity in the MT modeling. The modified RP MT modeling process is then used to quantify the macromolecular proton fraction (MMPF). The total water proton fraction (TWPF) is calculated as TWPF=1−MMPF. A multispoke acquisition strategy can be applied to the MT-Cones sequence to reduce scan time.

In the STAIR-Cones sequence, as illustrated in Panel B of FIG. 1, an Adiabatic Full Passage (AFP) pulse can be utilized to suppress long-T₂ water (e.g., intra/extracellular water) signals. For example, the AFP pulse can be utilized to invert the longitudinal magnetization of water components without significant compromise due to system B₀ and B₁ inhomogeneities. The repetition time (TR) of the STAIR-Cones sequence can be relatively short (e.g., 250 ms) to enable robust long-T₂ water signal suppression. Using a sufficiently short TR (e.g., 250 ms) in conjunction with an optimal inversion time (TI) in the STAIR-Cones sequence can enable the suppression of signals from long T₂ water components such as IEW and FW with a wide range of T₁s, allowing selective imaging of the fast-recovering short T₁ MW (essentially acting as a T₁ filter). Additionally, MW has a relatively short T₂ relaxation time (˜10 ms). The longitudinal magnetization of MW is partially inverted by the relatively long AFP pulse (e.g., 8.64 ms), allowing more signal recovery from MW during TI in STAIR-Cones. A multispoke acquisition strategy can be applied to the STAIR-Cones sequence to reduce scan time.

The highly T₂w-Cones sequence, as illustrated in Panel C of FIG. 1, can include four major features: (i) a magnetization reset module to generate a constant magnetization recovery, (ii) a T₂ preparation module with a long free decay time (e.g., 350 ms), (iii) an RF cycling or phase modulation scheme (e.g., the RF phase of the second 90° pulse alternates by 180° in adjacent TRs), and (iv) a Variable Flip Angle (VFA) technique to reduce signal variation along the multiple data acquisition spokes and improve the Signal-to-Noise Ratio (SNR) performance.

The PDw-Cones uses the sequence illustrated in Panel D of FIG. 1. A relatively low flip angle (e.g., 1°) for signal excitation and reduction of T₁-weighting, and is used with this sequence to mitigate the T₁ contrast. In addition, brain T₁ values are measured using a VFA-Cones technique with B₁ correction. The B₁ maps are measured by an actual flip angle Cones (AFI-Cones) sequence.

In each acquisition spoke, as illustrated in Panel D of FIG. 1, a slab-selective RF pulse is utilized for signal excitation followed by center-out spiral encoding. This spiral encoding, as illustrated in Panel E of FIG. 1, forms a 3D Cones trajectory that can efficiently cover k-space. In the embodiment described with respect to FIG. 1, each sequence uses a 3D Cones trajectory for measurement, which enhances compatibility of the results of each sequence (e.g., minimizes data offset between the resulting data). In some embodiments, other k-space trajectories are used for measurement, such as radial spokes (e.g., center-out radial spokes) and/or Cartesian sampling. In some embodiments, different trajectories are used for different measurements and/or sequences.

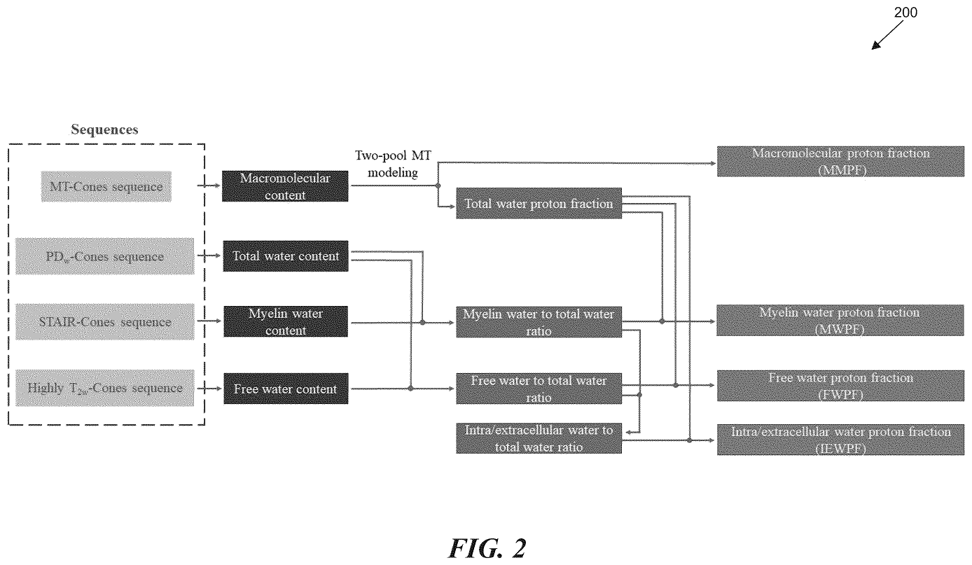

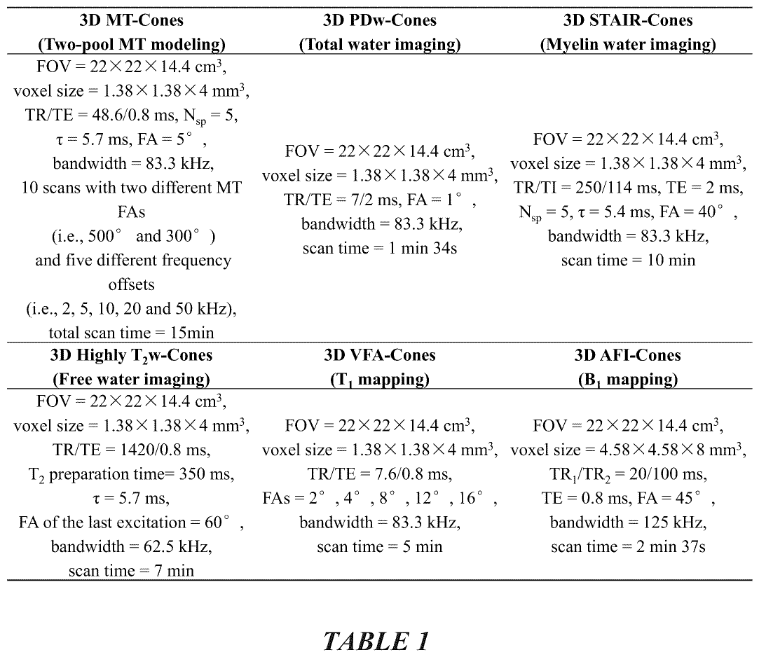

FIG. 2 shows a diagram depicting an example workflow 200 to quantify proton fractions (PFs) of macromolecules, MW, IW, EW, and FW in the brain, in accordance with the present technology. Macromolecular proton fraction (MMPF) and TW fraction are first estimated by two-pool MT modeling. The ratios of MW, IW, and EW to TW are calculated from STAIR-Cones, PDw-Cones, and T₂w-Cones images. With known TW fraction estimated by the MT modeling, the MW PF (MWPF), IW PF (IWPF), and EW PF (EWPF) are easily obtained.

The MW, TW, and FW images are directly generated by the STAIR-Cones, PDw-Cones, and highly T₂w-Cones scans, respectively. The MWF, defined as the content ratio between MW and TW, is calculated as MWF=MW/TW, taking into account the mixed T₁ and T₂* weighting terms from the STAIR-Cones and PDw-Cones sequences. The FWF, defined as the content ratio between FW and TW, is calculated in two steps. First, the signal ratio between the highly T₂w-Cones and PDw-Cones is determined. Then, since the FWF in the ventricular region should be 1, the FWF is estimated by normalizing the signal ratio map to the signal ratio from the ventricular region. This normalization step successfully eliminates the mixed T₁, T_{2*}, or T₂ weighting terms from the highly T₂w-Cones and PDw-Cones sequences.

With known MWF and FWF, the IEWF, defined as the content ratio between IEW and TW, can easily be obtained using:

$$IEWF = 1 - MWF - FWF , with MWF = \frac{MW}{TW} and FWF = \frac{FW}{TW} . \qquad [ 1 ]$$

The MMPF is directly obtained by two-pool MT modeling of a series of MT-Cones data. The TWPF can be easily calculated by TWPF=1−MMPF, which is defined as the ratio of TW to total proton content (i.e., TW+macromolecular proton (MMP)):

$$TWPF = \frac{TW}{TW + MMP} . \qquad [ 2 ]$$

Finally, with known values of MWF, FWF, and IEWF as well as TWPF, the MWPF, FWPF and IEWPF are easily obtained:

$$MWPF = MWF * TWPF , \qquad [ 3 ]$$

$$FWPF = FWF * TWPF , \qquad [ 4 ]$$

$$IEWPF = IEWF * TWPF . \qquad [ 5 ]$$

The proton exchange processes between MW and IEW have been considered in our STAIR-Cones sequence optimization and MWF calculation. Further incorporating MT into the signal modeling may improve the accuracy of MW imaging and our multicompartment modeling. The proposed simplified multicompartment model can be considered more robust and easier to translate into clinical practice compared to the complex four-pool MT modeling. The MT models with more than three pools are typically limited by their complexity, unreliable parameter estimation, and sensitivity to hardware imperfections and physiological noise, which affect the accurate quantification of the compartments and their exchange effects in in-vivo studies.

TABLE 1 provides a detailed sequence used in the study, describing detailed scan parameters of the comprehensive MR imaging protocol. In this study, ten healthy volunteers (aged 27±2 years old, six females) and five patients with MS (aged 55±11 years old, four females) were recruited and scanned. Written informed consent was obtained from each participant as approved by the institutional review board (IRB) of the University of California, San Diego, with registration number 201647. All participants underwent scanning with a 3T clinical scanner (MR750; GE Healthcare, Milwaukee, Wis), utilizing an 8-channel receive-only head coil for signal reception. The protocol's repeatability was assessed by scanning two healthy volunteers a total of three times each on different days. Table 1 provides detailed sequence parameters used in this study.

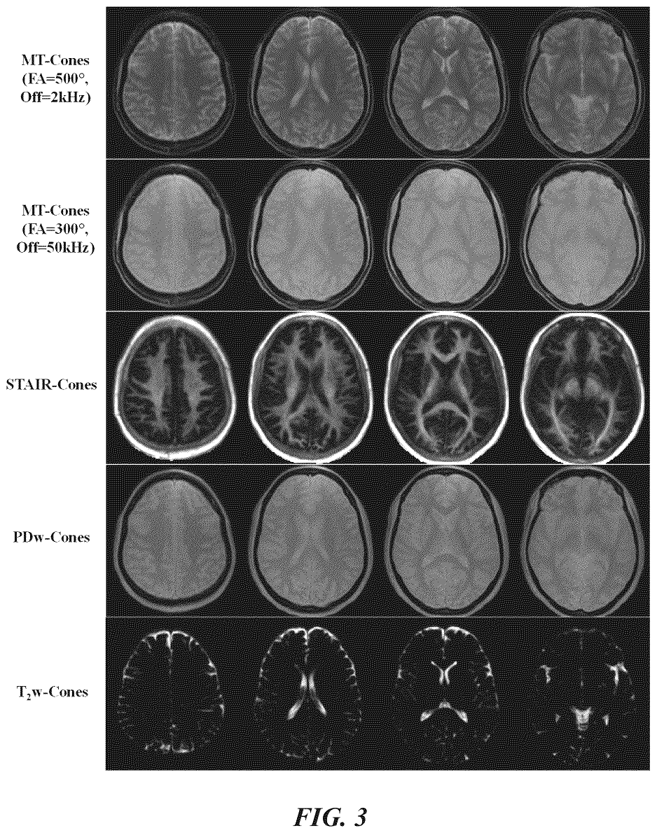

FIG. 3 shows the representative images acquired with MT-Cones, PDw-Cones, STAIR-Cones and T₂w-Cones sequences in a 22-year-old volunteer. Images with a higher MT FA and a smaller frequency offset show a stronger MT effect. As seen in the STAIR-Cones images, the white matter region has a much higher myelin water content than the gray matter region. Extracellular water is selectively imaged with the highly T₂w-Cones sequence, when signals of all the other water compartments are completely decayed. The STAIR-Cones images reveal a higher concentration of MW in WM regions than in GM regions. In the highly T₂w-Cones images, the extended T₂ preparation time (e.g., 350 ms) ensures complete decay of signals from MM, MW, and IEW (which have relatively short T₂s) while retaining signals from long T₂ CSF.

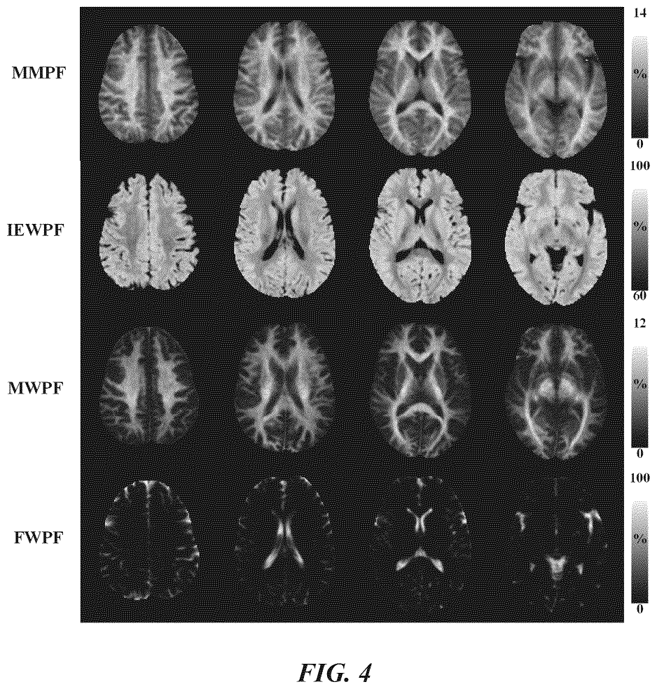

FIG. 4 shows the brain multicompartment mapping results, including example MMPF, MWPF, IWPF, and EWPF maps. The MMPF, MWPF, and IWPF in white matter range from 10 to 18%, 5 to 12%, and 65 to 75%, respectively. The MMPF, MWPF, and IWPF in gray matter range from 4 to 7%, 1.5 to 3%, and 81 to 88%, respectively. The EWPF ranges from 0 to 100%. As can be seen in these maps, the white matter shows much higher values of MMPF and MWPF than the gray matter. The gray matter has a higher IWPF than the white matter. Extracellular water mainly exists in non-white and gray matter regions, such as the ventricles and subarachnoid space.

Notably, WM exhibits much higher MMPF and MWPF values than GM, whereas GM demonstrates higher IEWPF values than WM. FW is predominantly present in the ventricles and subarachnoid space, and is largely absent from both WM and GM regions in a normal brain, with FWPF in both WM and GM ranging from 0 to 4%.

The repeatability analysis of the three scans done in each volunteer shows high ICC values for MMPF, MWPF, IEWPF, and FWPF measurements (Subject 1:0.98, 0.98, 0.98, and 0.88, respectively; Subject 2:0.99, 0.99, 0.99, and 0.90, respectively), indicating excellent repeatability of the comprehensive protocol.

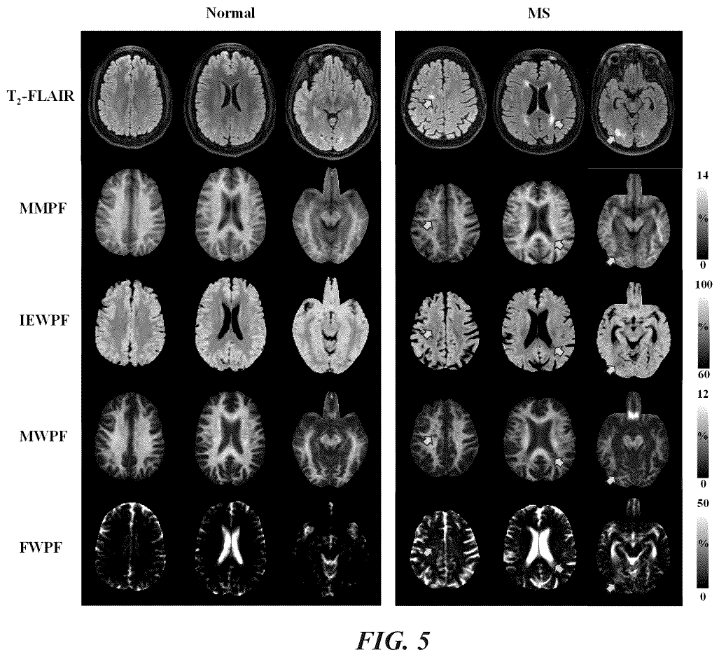

FIG. 5 shows MMPF, MWPF, IEWPF, and FWPF mapping comparison between a 26-year-old normal volunteer (left panel) and three MS patients of ages 68, 63, and 58 years from first to third columns in the right panel. T₂-FLAIR images are displayed in the first row of both panels for lesion localization. Lesions in the MS patients are shown by the yellow arrows. depicts PF maps acquired from a normal volunteer and three patients diagnosed with MS. Lesions were identified using T₂-FLAIR images. They are absent in the normal group but display their characteristic hyperintensities in the MS group. Lesions are distinguishable from surrounding ventricular, WM, and GM regions in all PF maps (highlighted by yellow arrows). They appear as hypointensities on the MMPF, IEWPF, and MWPF maps. Notably, on the FWPF map, lesions show mild hyperintensities, albeit less intense than FW within the ventricular region.

Neuroinflammatory and neurodegenerative diseases often manifest their pathological progression through compositional alterations. Particularly in MS, focal lesions, diffuse damage to myelin sheaths and axons, and the replacement of tissue by CSF are prominent features. Our technique shows that in MS patients, lesion MMPF, MWPF, and IEWPF values are notably lower than those observed in WM and GM regions in both normal volunteer and MS groups. Despite being localized in WM areas, lesion PFs closely resemble those of GM, indicating a significant degradation of myelin structure.

The MMPFs of NAWM are significantly lower than those of NWM, consistent with findings in previous neuropathological studies. The decrease in MM content in WM lesions may stem from neurodegeneration. The changes in lesions may be associated with pathological and inflammatory alterations around them, leading to adjacent NWM transitioning into NAWM.

NAWM shows a significant decrease in MWPF compared to NWM, while lesions have notably lower MWPF than both NWM and NAWM. This phenomenon may arise from diffuse neurodegeneration in MS brains, causing tissues adjacent to lesions to exhibit behavior akin to NAWM. These findings are consistent with prior studies demonstrating a widespread reduction in MWF of NAWM in MS patients compared to normal volunteers, with further decreases observed as the disease progresses.

Significant differences are observed in IEWPF measurements between NWM and NAWM, NWM and lesions, as well as between NAWM and lesions. These findings are consistent with prior research indicating an elevated level of extracellular water in MS patients compared to normal volunteers, possibly due to the breakdown of structural barriers affecting water motion. The considerably lower IEWPFs in MS lesions compared to NAWM may reflect structural damage. It is noteworthy that while clinically utilized T₂-FLAIR relies primarily on the T₂ contrast of the IEW for diagnosis, our study maps the PF of IEW.

Both WM and GM regions exhibit relatively low FWPFs in both the normal volunteer and MS groups, as free water predominantly occupies non-WM or GM regions, such as the ventricular system and subarachnoid space. Recent studies on MS lesions have demonstrated a transition in volume from lesion to CSF due to atrophy or lesion destruction. This finding is reflected in our results, where lesion FWPF measurements are significantly and markedly higher than those of both WM and GM in both groups. Moreover, FWPF measurements of NAWM and NAGM are significantly higher than those of the corresponding regions in NWM and NGM, respectively. This may be attributed to the aforementioned diffuse neurodegeneration and requires validation in future studies. The overall increase in FW content in MS brains may have implications for the interplay between different compartments throughout the course of disease progression.

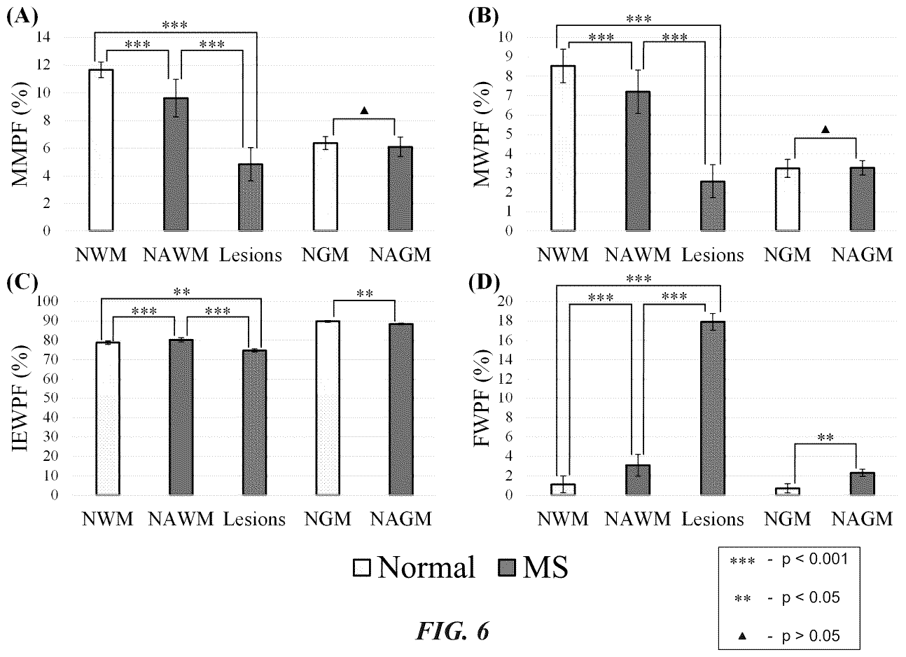

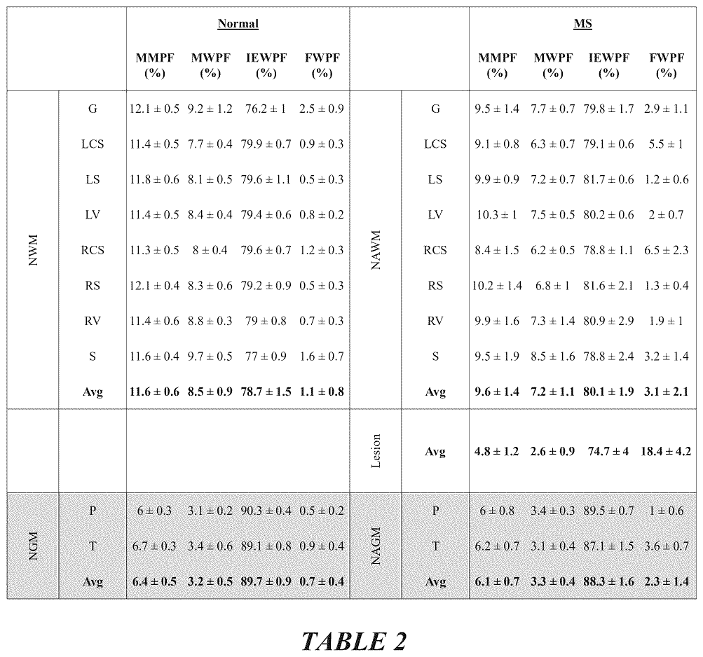

FIG. 6 depicts bar plots of averaged measured MMPF, MWPF, IEWPF, and FWPF values of NWM/NAWM, NGM/NAGM, and lesions from the ten normal volunteers and the five patients with MS. Independent t-test analysis was performed to investigate statistical differences of all the PF measurements between NWM and NAWM, between NWM and lesions, between NAWM and lesions, and between NGM and NAGM (“***” indicates p<0.001, “**” indicates p<0.05, and “▴” indicates p>0.05). NWM and NGM bars from the normal volunteers are depicted in dotted white. NAWM and NAGM bars from the patients with MS are depicted in gray and the Lesions bar is depicted in dotted gray. MMPF and MWPF measurements in relatively normal WM matter regions (e.g., NWM and NAWM) are notably higher than those in GM regions in both healthy volunteers and MS patients, consistent with WM's higher myelin content compared to GM. Moreover, significant differences in all four measurements, including MMPF, MWPF, IEWPF, and FWPF, are seen between NWM and NAWM, NWM and lesions, and NAWM and lesions. Significant differences between NGM and NAGM measurements are seen only in IEWPF and FWPF, but not in MMPF and MWPF.

TABLE 2 shows summarized measurements of MMPF, MWPF, IEWPF, and FWPF (mean±SD) in eight NWM/NAWM regions: G (genu) LCS (left centrum semiovale), LS (left subcortical WM), LV (left periventricular region), RCS (right centrum semiovale), RS (right subcortical WM), RV (right periventricular region), S (splenium), two NGM/NAGM regions: P (putamen), T (thalamus), and lesions in ten normal volunteers and five patients with MS.

### Method Flow

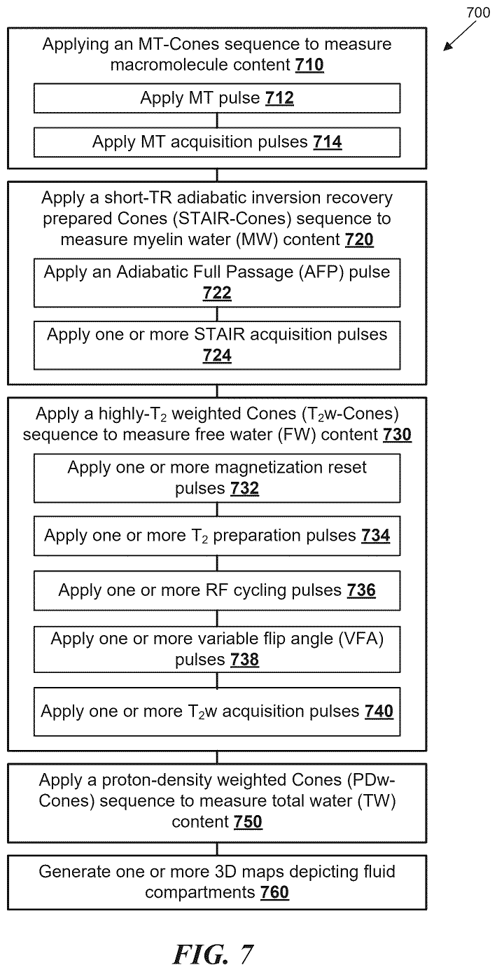

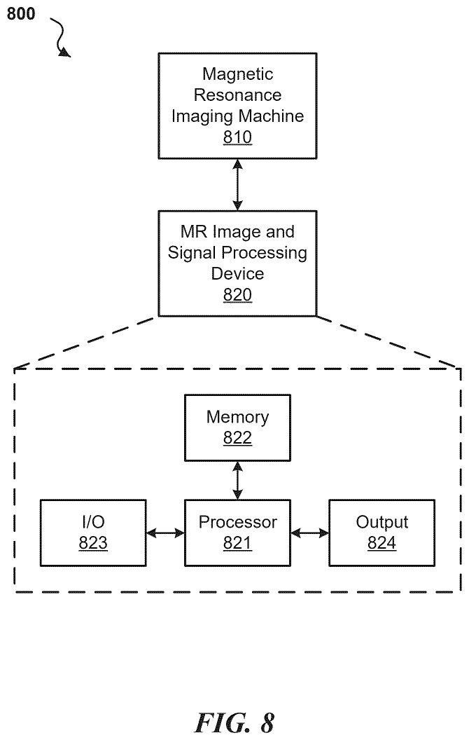

FIG. 7 shows a flow diagram illustrating an example method 700 for characterizing fluid compartments in tissue (e.g., brain tissue), in accordance with the present technology. The method 700 can include operations and/or calculations described with respect to FIGS. 1-6 above, Table 1, and/or Table 2. In some embodiments, the method 700 is performed by components of one or more computing systems and/or MRI systems (e.g., 3T clinical scanner), such as the example system 800 including the MRI machine 810 and the MR image and signal processing device 820, shown later in FIG. 8. Likewise, embodiments can include different and/or additional operations or can perform operations in different orders.

The method 700 can include, at operation 710, applying a magnetization transfer prepared Cones (MT-Cones) sequence to generate first magnetic resonance (MR) data. As shown in FIG. 7, the first MR data can be used to measure and/or determine a macromolecule (MM) content (e.g., MM intensity data). In some embodiments, the first MR data is used to measure intensity data for MM content, intracellular/extracellular water (IEW) content, and/or free water (FW) content. The MT-Cones sequence can include a Fermi-shaped MT pulse to generate MT contrast. Each pulse can be followed by one or more Cones measurements. A series of MT-Cones data can be acquired using different MT parameters, such as power or frequency offset. In some embodiments, the first MR data is used to determine macromolecular content of the tissue by applying two-pool MT modeling to the first MR data. For example, T₁ values can be measured by an AFI-VFA-Cones technique and used for two-pool MT modeling.

As an illustrative example of applying the MT-Cones sequence, the method 700 can include, at operation 712, applying an MT pulse. In some embodiments, the MT pulse is a Fermi-shaped MT pulse. The method 700 can include, at operation 714, applying one or more acquisition pulses (e.g., MT acquisition pulses). Each MT acquisition pulse can be applied in accordance with a Cones technique (e.g., where each MT acquisition pulse generates data for points on a cone in k-space). In some embodiments, the method 700 includes applying an MT sequence that uses an alternate technique to efficiently cover k-space (e.g., alternatively to and/or in addition to applying the MT-Cones sequence at operation 710), such as a center-out radial technique and/or Cartesian sampling.

In some embodiments, applying the MT-Cones sequence includes using an actual flip angle and variable flip angle Cones (AFI-VFA-Cones) technique (e.g., to measure tissue T₁ values), such as applying actual flip angle (AFI) pulses and/or variable flip angle (VFA) pulses (e.g., as part of the one or more MT acquisition pulses). In some embodiments, the first MR data generated from applying the MT-Cones sequence is used to perform two-pool MT modeling. In some implementations, the method 700 includes applying multiple MT-Cones sequences, which can utilize different MT parameters, such as powers and/or frequency offsets. For example, the method 700 can include applying 10 MT-Cones sequences (which can have the same and/or different parameters from each other).

The method 700 can include, at operation 720, applying a short-repetition-rate (short-TR) adiabatic inversion-recover prepared Cones (STAIR-Cones) sequence to measure second MR data. As shown in FIG. 7, the second MR data can be used to measure and/or determine a myelin water (MW) content (e.g., MW intensity data). The STAIR-Cones sequence has a short repetition time (TR), such as 250 ms or less.

As an illustrative example of applying the STAIR-Cones sequence, the method 700 can include, at 722, applying an Adiabatic Full Passage (AFP) pulse. The AFP pulse can be configured to suppress long T₂ water, such as intra/extracellular water (IEW). In some embodiments, the AFP pulse is relatively long, such as 8.64 ms or longer. The method 700 can include, at 724, applying one or more acquisition pulses (e.g., STAIR acquisition pulses). Each STAIR acquisition pulse can be applied in accordance with a Cones technique (e.g., where each STAIR acquisition pulse generates data for points on a cone in k-space). In some embodiments, the method 700 includes applying the STAIR-Cones sequence multiple times.

The method 700 can include, at operation 730, applying a highly T₂-weighted Cones (T₂w-Cones) sequence to generate third MR data. As illustrated in FIG. 7, the third MR data can be used to measure and/or determine a free water (FW) content (e.g., FW intensity data).

As an illustrative example of applying the T₂w-Cones sequence, the method 700 can include, at operation 732, applying one or more magnetization reset pulses. The magnetization reset pulses can generate a constant magnetization recovery. The method 700 can include, at operation 734, applying one or more T₂ preparation pulses. The T₂ preparation pulses can be associated with a long free decay time, such as 350 ms or longer. The method 700 can include, at operation 736, applying one or more radio frequency (RF) cycling pulses. The RF cycling pulses can be part of an RF cycling and/or phase modulation scheme. For example, in an RF cycling and/or phase modulation scheme in which each repetition includes at least two 90° pulses, the 90° pulses can be opposite in sequential repetitions (e.g., alternating by 180° in adjacent repetitions).

Continuing the illustrative example above, the method 700 can include, at operation 738, applying one or more variable flip angle (VFA) pulses. Each VFA pulse can be configured to reduce signal variation along data acquisition spokes (e.g., measured by acquisition pulses) and/or improve a signal-to-noise ratio (SNR) performance. The method 700 can include, at operation 740, applying one or more acquisition pulses (e.g., T₂w acquisition pulses). Each T₂w acquisition pulse can be applied in accordance with a Cones technique (e.g., where each T₂w acquisition pulse generates data for points on a cone in k-space). In some embodiments, the method 700 includes applying multiple T₂w-Cones sequences. In some embodiments, the method 700 includes applying a T₂w sequence that uses an alternate technique to efficiently cover k-space (e.g., alternatively to and/or in addition to applying the T₂w-Cones sequence at operation 730), such as a center-out radial technique and/or Cartesian sampling.

The method 700 can include, at operation 750, applying a proton-density weighted Cones (PDw-Cones) sequence to generate a fourth MR data. As illustrated in FIG. 7, the fourth MR data can be used to measure and/or determine a total water (TW) content (e.g., TW intensity data). The PDw-Cones sequence can include a relatively low flip angle (e.g., 1°) for signal excitation and reduction of T₁-weighting. In some embodiments, T₁ values are measured using a VFA-Cones technique, which can include a B₁ correction. The B₁ correction can be based on a B₁ map measured using an AFI-Cones sequence. In some embodiments, the method 700 includes applying multiple PDw-Cones sequences.

Applying the PDw-Cones sequence can include applying acquisition pulses (e.g., PDw acquisition pulses), where each PDw acquisition pulse can be applied in accordance with a Cones technique (e.g., where each PDw acquisition pulse generates data for points on a cone in k-space). In some embodiments, the method 700 includes applying a PDw sequence that uses an alternate technique to efficiently cover k-space (e.g., alternatively to and/or in addition to applying the PDw-Cones sequence at operation 750), such as a center-out radial technique and/or Cartesian sampling.

The method 700 can include, at operation 760, generating one or more 3D maps depicting fluid compartments (and/or tissue components, such as brain tissue components). Generating the 3D maps can include generating intensity data for one or more of MM, MW, TW, and/or FW. Generating the 3D maps can include computing water fraction data, such as a macromolecular proton fraction (MMPF), a myelin water proton fraction (MWPF), an intra/extracellular water proton fraction (IEWPF), and/or a free water proton fraction (FWPF).

In some embodiments, the method 700 includes determining (e.g., calculating) values for water fractions of a water fraction type set (e.g., including MMPF, MWPF, IEWPF, and/or FWPF). The values can include at least one value for a white matter (WM) region of the brain and for a gray matter (GM) region of the brain. That is, each water fraction type can be associated with at least one value measured in WM and/or measured in GM. These values can be used to detect lesions, such as MS lesions.

It is contemplated that the operations or descriptions of FIG. 7 may be used with any other embodiment of this disclosure. In addition, the operations and descriptions described in relation to FIG. 7 may be done in alternative orders or in parallel to further the purposes of this disclosure. For example, each of these operations may be performed in any order, multiple times, in parallel, or simultaneously to reduce the time associated with performing the method 700. Furthermore, it should be noted that any of the devices or equipment discussed in relation to the other figures or otherwise disclosed herein could be used to perform one or more of the operations in FIG. 7.

### MRI System

FIG. 8 shows a diagram of an example embodiment of a system 800 that includes a magnetic resonance imaging (MRI) machine 810 in communication with an MR image and signal processing device 820, e.g., which can be used to control the MRI machine and analyze obtained data to effect the image data collecting protocol to produce quantitative data in accordance with the disclosed 3D MRI method.

The MRI machine 810 can be used in the system 800 to implement a MRI-based characterization process in accordance with example embodiments of the 3D MRI method of the present technology under the control of the example MR image and signal processing device 820. MRI machine 810 can include various types of MRI systems, which can perform at least one of a multitude of MRI scans that can include, but are not limited to, T₁-weighted MRI scans, T_{1ρ} MRI scans, T₂-weighted MRI scans, T₂*-weighted MRI scans, spin (proton (1H)) density weighted MRI scans, diffusion tensor (DT) and diffusion weighted imaging (DWI) MRI scans, magnetization transfer (MT) MRI scans, real-time MRI, functional MRI (fMRI) and related techniques such as arterial spin labeling (ASL), among other MRI techniques.

The MR image and signal processing device 820 can include a processor 821 that can be in communication with a memory unit 822, an input/output (I/O) unit 823, and/or an output unit 824. The MR image and signal processing device 820 can be implemented as one of various data processing systems, such as a personal computer (PC), laptop, and mobile computing device such as a smartphone, tablet and/or wearable computing device. In some implementations, the MR image and signal processing device 820 is embodied on one or more computing devices in a computer system or communication network accessible via the Internet (referred to as “the cloud”), e.g., including servers and/or databases in the cloud.

The processor 821 is configured to process data, and the memory unit 822 is in communication with the processor 821 to store and/or buffer the data. To support various functions of the MR image and signal processing device 820, the processor 821 can be included to interface with and control operations of other components of the MR image and signal processing device 820, such as the I/O unit 823 and/or the output unit 824. The processor 821 can include one or more processors, e.g., including but not limited to microprocessors such as a central processing unit (CPU), microcontrollers, or the like.

The memory unit 822 can include and store processor-executable code, which when executed by the processor, configures the MR image and signal processing device 820 to perform various operations, e.g., such as receiving information, commands, and/or data, processing information and data, and transmitting or providing information/data to another device. The memory unit 822 can store other information and data, such as instructions, software, values, images, and other data processed or referenced by processor 821. For example, various types of Random Access Memory (RAM) devices, Read Only Memory (ROM) devices, Flash Memory devices, and other suitable storage media can be used to implement storage functions of memory unit 822. The memory unit 822 can store MRI data and information, which can include subject MRI image data including spatial and spectral data, MRI machine system parameters, data processing parameters, and processed parameters and data that can be used in the implementation of MR signal and data processing techniques, including 3D MRI techniques in accordance with the disclosed technology. The memory unit 822 can store data and information that can be used to implement a MRI-based imaging and signal characterization method, e.g., including one or more algorithms for implementing a 3D MRI method, and store data and information that can be generated from an algorithm and/or model of the 3D MRI-based protocol in accordance with the disclosed technology.

In some implementations, the MR image and signal processing device 820 includes the I/O unit 823 to interface the processor 821 and/or memory unit 822 to other modules, units or devices associated with the system 800, and/or external devices. The I/O unit 823 can connect to an external interface, source of data storage, or display device. Various types of wired or wireless interfaces compatible with typical data communication standards, such as Universal Serial Bus (USB), IEEE 1394 (FireWire), Bluetooth, Bluetooth low energy (BLE), ZigBee, IEEE 802.11, Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), 3G/4G/LTE/5G/6G cellular communication methods, and parallel interfaces, can be used to implement I/O unit 823. In some implementations, for example, the MR image and signal processing device 820 includes a wireless communications unit, e.g., such as a transmitter (Tx) or a transmitter/receiver (Tx/Rx) unit. The I/O unit 823 can interface the processor 821 and memory unit 822 with the wireless communications unit to utilize various types of wireless interfaces, such as the examples described above. The I/O unit 823 can interface with other external interfaces, sources of data storage, and/or visual or audio display devices, etc. to retrieve and transfer data and information that can be processed by the processor 821, stored in the memory unit 822, or exhibited on an output unit of a user device (e.g., display screen of a computing device) or an external device.

To support various functions of the MR image and signal processing device 820, the output unit 824 can be used to exhibit data implemented by the MR image and signal processing device 820. The output unit 824 can include various types of display, speaker, or printing interfaces to implement output functionalities the system 800. In some embodiments, for example, the output unit 824 can include cathode ray tube (CRT), light emitting diode (LED), or liquid crystal display (LCD) monitor or screen as a visual display. In some examples, the output unit 824 can include toner, liquid inkjet, solid ink, dye sublimation, inkless (such as thermal or UV) printing apparatuses to implement some output modalities of the output unit 824. In some examples, the output unit 824 can include various types of audio signal transducer apparatuses. The output unit 824 can exhibit data and information, such as patient diagnostic data, MRI machine system information, partially processed information during implementations of the 3D MRI method, and/or fully-processed information during implementations of the 3D MRI method.

### EXAMPLES

In some embodiments in accordance with the disclosed technology (example A1), a method for magnetic resonance imaging for characterizing fluid compartments in brain tissue includes acquiring and processing magnetic resonance (MR) data from a tissue using a magnetic resonance imaging (MRI) system; the MRI data including magnetization transfer prepared Cones (MT-Cones), short-TR adiabatic inversion-recovery prepared Cones (STAIR-Cones), proton-density weighted Cones (PDw-Cones), and highly T₂-weighted Cones (T₂w-Cones); and creating a three-dimensional (3D) map depicting tissue components and fluid components of the tissue based on the acquired and processed MR data.

Example A2 includes the method of example A1 or any of examples A1-A15, wherein the magnetization transfer prepared Cones (MT-Cones) provides two-pool MT modeling and macromolecular content of the tissue.

Example A3 includes the method of example A1 or any of examples A1-A15, wherein the short-TR adiabatic inversion-recovery prepared Cones (STAIR-Cones) provides myelin water imaging of the tissue.

Example A4 includes the method of example A1 or any of examples A1-A15, wherein the proton-density weighted Cones (PDw-Cones) provides total water imaging of the tissue.

Example A5 includes the method of example A1 or any of examples A1-A15, wherein the highly T₂-weighted Cones (T₂w-Cones) provides free water imaging of the tissue.

Example A6 includes the method of example A1 or any of examples A1-A15, wherein the MR data is processed to produce images of macromolecules, intra/extracellular water, and free water content in the tissue.

Example A7 includes the method of example A6 or any of examples A1-A15 wherein the MR data is further processed to produce images of myelin water in the tissue.

Example A8 includes the method of example A1 or any of examples A1-A15, wherein the processing of the MR data comprises using a 3T clinical scanner.

Example A9 includes the method of example A1 or any of examples A1-A15, wherein the tissue is a brain tissue.

Example A10 includes the method of example A1 or any of examples A1-A15, wherein the MR data includes brain macromolecular proton fraction (MMPF), myelin water proton fraction (MWPF), intra/extracellular water proton fraction (IEWPF), and free water proton fraction (FWPF) values.

Example A11 includes the method of example A10 or any of examples A1-A15, wherein the brain macromolecular proton fraction (MMPF), myelin water proton fraction (MWPF), intra/extracellular water proton fraction (IEWPF), and free water proton fraction (FWPF) values are measured in white matter (WM) and gray matter (GM).

Example A12 includes the method of example A10 or any of examples A1-A15, wherein the brain macromolecular proton fraction (MMPF), myelin water proton fraction (MWPF), intra/extracellular water proton fraction (IEWPF), and free water proton fraction (FWPF) values are measured in a brain lesion.

Example A13 includes the method of example A10 or any of examples A1-A15, wherein the brain macromolecular proton fraction (MMPF), myelin water proton fraction (MWPF), intra/extracellular water proton fraction (IEWPF), and free water proton fraction (FWPF) values are measured in a neurological disorder.

Example A14 includes the method of example A10 or any of examples A1-A15, wherein the brain macromolecular proton fraction (MMPF), myelin water proton fraction (MWPF), intra/extracellular water proton fraction (IEWPF), and free water proton fraction (FWPF) values are measured in a Multiple Sclerosis (MS) lesion.

Example A15 includes the method of example A10 or any of examples A1-A14, wherein the method is able to discern between a healthy tissue and a pathological tissue.

In some embodiments in accordance with the disclosed technology (example A16), a system for performing the MRI method of any of examples A1-AX, including an MRI acquisition machine; and a data processing device comprising a processor and a memory, wherein the memory is configured to store instructions of a computer program product, which when executed by the processor, causes the system to perform the MRI method of any of examples A1-AX.

Example A17 includes the system of example A16, wherein the quantification is done by a 3T clinical scanner.

In some embodiments in accordance with the disclosed technology (example B1), a method for magnetic resonance imaging (MRI) for characterizing tissue components and fluid compartments in brain includes applying a magnetization transfer prepared Cones (MT-Cones) sequence to generate first magnetic resonance (MR) data, wherein the MT-Cones sequence comprises: an MT pulse, and one or more MT acquisition pulses, each MT acquisition pulse measuring data on a cone in k-space; applying a short-repetition-rate (short-TR) adiabatic inversion-recovery prepared Cones (STAIR-Cones) sequence to generate second MR data, wherein the STAIR-Cones sequence comprises: an Adiabatic Full Passage (AFP) pulse, and one or more STAIR acquisition pulses, each STAIR acquisition pulse measuring data on a cone in k-space; applying a highly T₂-weighted Cones (T₂w-Cones) sequence to generate third MR data, wherein the T₂w-Cones sequence comprises: one or more magnetization reset pulses, one or more T₂ preparation pulses, one or more radio frequency (RF) cycling pulses, one or more variable flip angle (VFA) pulses, and one or more T₂w acquisition pulses, each T₂w acquisition pulse measuring data on a cone in k-space; applying a proton-density weighted Cones (PDw-Cones) sequence to generate fourth MR data; and generating, using at least the first MR data, the second MR data, the third MR data, and the fourth MR data, one or more three-dimensional (3D) maps depicting tissue components and fluid compartments of the brain.

Example B2 includes the method of example B1 or any of examples B1-B19, wherein the MT pulse of the MT-Cones sequence is a Fermi-shaped MT pulse.

Example B3 includes the method of example B1 or any of examples B1-B19, wherein the one or more MT acquisition pulses comprises an actual flip angle (AFI) or variable flip angle (VFA) pulse.

Example B4 includes the method of example B1 or any of examples B1-B19, comprising: applying two-pool MT modeling to the first MR data to determine macromolecular content of the tissue.

Example B5 includes the method of example B1 or any of examples B1-B19, wherein the first MR data comprises intensity data for one or more of: macromolecule (MM) content, intracellular/extracellular water content (IEW), or free water (FW) content.

Example B6 includes the method of example B1 or any of examples B1-B19, wherein a repetition time of the STAIR-Cones sequence is 250 ms or less.

Example B7 includes the method of example B1 or any of examples B1-B19, wherein the second MR data comprises intensity data for myelin water (MW) content.

Example B8 includes the method of example B1 or any of examples B1-B19, wherein the one or more T₂ preparation pulses is configured to have a decay time of 350 ms or more.

Example B9 includes the method of example B1 or any of examples B1-B19, wherein the third MR data comprises intensity data for free water (FW) content.

Example B10 includes the method of example B1 or any of examples B1-B19, wherein the fourth MR data comprises intensity data for total water (TW) content.

Example B11 includes the method of example B1 or any of examples B1-B19, comprising: generating one or more images of myelin water (MW) using one or more of: the first MR data, the second MR data, the third MR data, or the fourth MR data.

Example B12 includes the method of example B1 or any of examples B1-B19, comprising: processing, using a 3T clinical scanner, one or more of: the first MR data, the second MR data, the third MR data, or the fourth MR data.

Example B13 includes the method of example B1 or any of examples B1-B19, comprising: calculating, using at least the first MR data, the second MR data, the third MR data, and the fourth MR data, a proton fraction value set, wherein the proton fraction value set is associated with a proton fraction type set comprising a brain macromolecular proton fraction (MMPF), a myelin water proton fraction (MWPF), an intra/extracellular water proton fraction (IEWPF), and a free water proton fraction (FWPF); wherein each value of the proton fraction value set is associated with a proton fraction type of the proton fraction type set.

Example B14 includes the method of example B13 or any of examples B1-B19, wherein the proton fraction value set comprises a first FWPF value, and wherein the FWPF value is calculated at least in part by: determining, from the third MR data, a first free water (FW) value and a second FW value, determining, from the fourth MR data, a first total water (TW) value and a second TW value, determining a first second FWPF value by taking a ratio of the first FW value and the first TW value, wherein the first FW value and the first TW value are measured in a ventricular region; normalizing the second FWPF value to equal 1, and normalizing, with respect to the normalized second FWPF value, a ratio of the second FW value and the second TW value to calculate the first FWPF value.

Example B15 includes the method of example B13 or any of examples B1-B19, wherein each proton fraction type of the proton fraction type is associated with a respective first value and a second value; wherein the respective first value is measured in white matter (WM); and wherein the respective second value is measured in gray matter (GM).

Example B16 includes the method of example B13 or any of examples B1-B19, wherein each proton fraction type is associated with a respective value measured in a brain lesion.

Example B17 includes the method of example B16 or any of examples B1-B19, wherein the brain lesion is a Multiple Sclerosis (MS) lesion.

Example B18 includes the method of example B13 or any of examples B1-B19, wherein each proton fraction type is associated with a respective value measured in a neurological disorder.

Example B19 includes the method of example B13 or any of examples B1-B18, comprising: determining, using the proton fraction value set, if a tissue portion of the brain tissue is either healthy or pathological.

In some embodiments in accordance with the disclosed technology (example B20), a magnetic resonance imaging (MRI) system includes an MRI acquisition machine; and a data processing device comprising a processor and a memory, wherein the memory is configured to store instructions of a computer program product, which when executed by the processor, causes the system to: apply a magnetization transfer prepared Cones (MT-Cones) sequence to generate first magnetic resonance (MR) data; apply a short-repetition-rate (short-TR) adiabatic inversion-recovery prepared Cones (STAIR-Cones) sequence to generate second MR data; apply a highly T₂-weighted Cones (T₂w-Cones) sequence to generate third MR data; and apply a proton-density weighted Cones (PDw-Cones) sequence to generate fourth MR data; generate, using at least the first MR data, the second MR data, the third MR data, and the fourth MR data, one or more three-dimensional (3D) maps depicting fluid compartments of tissue.

Example B21 includes the system of example B20 or any of examples B20-B24, comprising a 3T clinical scanner, wherein the instructions cause the system to apply, using the 3T clinical scanner, at least one of: the MT-Cones sequence, the STAIR-Cones sequence, the T₂w-Cones sequence or the PDw-Cones sequence.

Example B22 includes the system of example B20 or any of examples B20-B24, wherein the instructions cause the system to: calculate, using at least the first MR data, the second MR data, the fourth MR data, and the third MR data, a proton fraction value set, wherein the proton fraction value set is associated with a proton fraction type set comprising a brain macromolecular proton fraction (MMPF), a myelin water proton fraction (MWPF), an intra/extracellular water proton fraction (IEWPF), and a free water proton fraction (FWPF), and wherein each value of the proton fraction value set is associated with a proton fraction type of the proton fraction type set.

Example B23 includes the system of example B20 or any of examples B20-B24, wherein each proton fraction type of the proton fraction type is associated with a respective first value and a second value; wherein the respective first value is measured in white matter (WM); and wherein the respective second value is measured in gray matter (GM).

Example B24 includes the system of example B20 or any of examples B20-B23, wherein each proton fraction type is associated with a respective value measured in a brain lesion.

### CONCLUSION

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1. A method for magnetic resonance imaging (MRI) for characterizing tissue components and fluid compartments in brain, the method comprising:
applying a magnetization transfer prepared Cones (MT-Cones) sequence to generate first magnetic resonance (MR) data, wherein the MT-Cones sequence comprises:
an MT pulse, and
one or more MT acquisition pulses, each MT acquisition pulse measuring data on a cone in k-space;
applying a short-repetition-rate (short-TR) adiabatic inversion-recovery prepared Cones (STAIR-Cones) sequence to generate second MR data, wherein the STAIR-Cones sequence comprises:
an Adiabatic Full Passage (AFP) pulse, and
one or more STAIR acquisition pulses, each STAIR acquisition pulse measuring data on a cone in k-space;
applying a highly T₂-weighted Cones (T₂w-Cones) sequence to generate third MR data, wherein the T₂w-Cones sequence comprises:
one or more magnetization reset pulses,
one or more T₂ preparation pulses,
one or more radio frequency (RF) cycling pulses,
one or more variable flip angle (VFA) pulses, and
one or more T₂w acquisition pulses, each T₂w acquisition pulse measuring data on a cone in k-space;
applying a proton-density weighted Cones (PDw-Cones) sequence to generate fourth MR data; and
generating, using at least the first MR data, the second MR data, the third MR data, and the fourth MR data, one or more three-dimensional (3D) maps depicting tissue components and fluid compartments of the brain.

2. The method of claim 1, wherein the MT pulse of the MT-Cones sequence is a Fermi-shaped MT pulse.

3. The method of claim 1, wherein the one or more MT acquisition pulses comprises an actual flip angle (AFI) or variable flip angle (VFA) pulse.

4. The method of claim 1, comprising:
applying two-pool MT modeling to the first MR data to determine macromolecular content of the tissue.

5. The method of claim 1, wherein the first MR data comprises intensity data for one or more of: macromolecule (MM) content, intracellular/extracellular water content (IEW), or free water (FW) content.

6. The method of claim 1, wherein a repetition time of the STAIR-Cones sequence is 250 ms or less.

7. The method of claim 1, wherein the second MR data comprises intensity data for myelin water (MW) content.

8. The method of claim 1, wherein the one or more T₂ preparation pulses is configured to have a decay time of 350 ms or more.

9. The method of claim 1, wherein the third MR data comprises intensity data for free water (FW) content.

10. The method of claim 1, wherein the fourth MR data comprises intensity data for total water (TW) content.

11. The method of claim 1, comprising:
generating one or more images of myelin water (MW) using one or more of: the first MR data, the second MR data, the third MR data, or the fourth MR data.

12. The method of claim 1, comprising:
processing, using a 3T clinical scanner, one or more of: the first MR data, the second MR data, the third MR data, or the fourth MR data.

13. The method of claim 1, comprising:
calculating, using at least the first MR data, the second MR data, the third MR data, and the fourth MR data, a proton fraction value set,
wherein the proton fraction value set is associated with a proton fraction type set comprising a brain macromolecular proton fraction (MMPF), a myelin water proton fraction (MWPF), an intra/extracellular water proton fraction (IEWPF), and a free water proton fraction (FWPF);
wherein each value of the proton fraction value set is associated with a proton fraction type of the proton fraction type set.

14. The method of claim 13, wherein the proton fraction value set comprises a first FWPF value, and wherein the FWPF value is calculated at least in part by:
determining, from the third MR data, a first free water (FW) value and a second FW value,
determining, from the fourth MR data, a first total water (TW) value and a second TW value,
determining a first second FWPF value by taking a ratio of the first FW value and the first TW value,
wherein the first FW value and the first TW value are measured in a ventricular region;
normalizing the second FWPF value to equal 1, and
normalizing, with respect to the normalized second FWPF value, a ratio of the second FW value and the second TW value to calculate the first FWPF value.

15. The method of claim 13,
wherein each proton fraction type of the proton fraction type is associated with a respective first value and a second value;
wherein the respective first value is measured in white matter (WM); and
wherein the respective second value is measured in gray matter (GM).

16. The method of claim 13, wherein each proton fraction type is associated with a respective value measured in a brain lesion.

17. The method of claim 16, wherein the brain lesion is a Multiple Sclerosis (MS) lesion.

18. The method of claim 13, wherein each proton fraction type is associated with a respective value measured in a neurological disorder.

19. The method of claim 13, comprising:
determining, using the proton fraction value set, if a tissue portion of the brain tissue is either healthy or pathological.

20. A magnetic resonance imaging (MRI) system, comprising:
an MRI acquisition machine; and
a data processing device comprising a processor and a memory, wherein the memory is configured to store instructions of a computer program product, which when executed by the processor, causes the system to:
apply a magnetization transfer prepared Cones (MT-Cones) sequence to generate first magnetic resonance (MR) data;
apply a short-repetition-rate (short-TR) adiabatic inversion-recovery prepared Cones (STAIR-Cones) sequence to generate second MR data;
apply a highly T₂-weighted Cones (T₂w-Cones) sequence to generate third MR data; and
apply a proton-density weighted Cones (PDw-Cones) sequence to generate fourth MR data;
generate, using at least the first MR data, the second MR data, the third MR data, and the fourth MR data, one or more three-dimensional (3D) maps depicting fluid compartments of tissue.

21. The system of claim 20, comprising:
a 3T clinical scanner,
wherein the instructions cause the system to apply, using the 3T clinical scanner, at least one of: the MT-Cones sequence, the STAIR-Cones sequence, the T₂w-Cones sequence or the PDw-Cones sequence.

22. The system of claim 20, wherein the instructions cause the system to:
calculate, using at least the first MR data, the second MR data, the fourth MR data, and the third MR data, a proton fraction value set,
wherein the proton fraction value set is associated with a proton fraction type set comprising a brain macromolecular proton fraction (MMPF), a myelin water proton fraction (MWPF), an intra/extracellular water proton fraction (IEWPF), and a free water proton fraction (FWPF), and
wherein each value of the proton fraction value set is associated with a proton fraction type of the proton fraction type set.

23. The system of claim 20,
wherein each proton fraction type of the proton fraction type is associated with a respective first value and a second value;
wherein the respective first value is measured in white matter (WM); and
wherein the respective second value is measured in gray matter (GM).

24. The system of claim 20, wherein each proton fraction type is associated with a respective value measured in a brain lesion.
